(19) United States
(12) Patent Application Publication
(10) Pub. No.: US 2004/0067571 A1
(43) Pub. Date: Apr. 8, 2004

(54) Process for preparing cellulase and protease enzyme complex cud liquor from cattle and buffaloes cud for biodegradation of organic materials cud for biodegredation of organic materials

(76) Inventor: Gopal Krishna, Faridabad (ID)

Correspondence Address: Sidley Austin Brown & Wood, 1501 K Street N W, Washington, DC, 20005, US

(21) Appl. No.: 10/398,169

(22) PCT Filed: Sep. 27, 2002
(86) PCT No.: PCT/IN02/00197

(30) Foreign Application Priority Data
Jan. 31, 2002 (IN) 82/DEL/02

(51) Int. Cl. C12N 9/50; A61K 38/43

(52) U.S. Cl. 435/219; 424/94.1

(57) ABSTRACT

The present invention provides a process of preparing cellulase and protease enzyme complex cud liquor from cattle and buffaloes cud for biodegradation of organic materials. The process comprises feeding cattle/buffaloes green leguminous/non-leguminous fodder ad libitum. The fodder is withdrawn from said cattle/buffaloes after 2-3 hours and wait till cattle/buffalo is ruminating. Thereafter the fodder loop is inserted in the mouth of said cattle/buffaloes and said loop is allowed to stay in the mouth till the cud is embedded on said loop. The above steps are repeated with separate fodder loops and different cattle/buffaloes. The said loops are collected from the mouth of said cattle/buffaloes, and the loops embedded with cud are dipped in at least one litre buffer nutrient solution to obtain cellulase and protease concentrate.

### FIELD OF THE INVENTION

The present invention relates to a process to prepare cellulase and protease enzyme complex cud liquor from cattle and buffaloes cud for biodegradation of organic materials.

### BACKGROUND OF THE INVENTION

For studying biodegradation of Agro Industrial byproducts (organic materials), rumen liquor based in vitro system is commonly used through out the world. The prerequisite of rumen liquor collection in this method needs modified “RUMEN FISTULATED” cattle and buffaloes and such surgical operation “RUMEN FISTULATION” is a cruel act under the Prevention of Cruelty to Animal Act, 1960. An alternative method of rumen liquor based biodegradation is in situ method, where Agro Industrial byproducts is placed in the Nylon bag and inserted through the rumen fistula in the rumen fermentation vat (Orskov and MacDonald, 1979). There is a wide variation of end biodegradation results (in terms of In vitro dry matter and crude protein disappearance and degradability) from one series to another or from one laboratory to the other, even after vigorously standardizing the diet of the animals, the sampling conditions and the inoculum preparation.

It is a well-established fact that only enzyme based biodegradation vessel could yield reproducible and fermentation results. Throughout world at present crude fungal cellulase preparation (Trichoderma viride) manufactured by Sigma catalogue no. C-9422, located at USA/European subcontinent, is used as this enzyme has inherent nature of cellulolytic and proteolytic activity but India could not afford such high cost foreign exchange involved commodity for use in biodegradation fermentation vessel.

### FIELD OF INVENTION

The present invention relates to a process to prepare cellulase and protease enzyme complex cud liquor from cattle and buffaloes cud for biodegradation of organic materials.

### BACKGROUND OF THE INVENTION

For studying biodegradation of Agro Industrial byproducts (organic materials), rumen liquor based in vitro system (Two stage method of Tilley and Terry, 1963) is commonly used through out the world. The prerequisite of rumen liquor collection in this method needs modified “RUMEN FISTULATED” cattle and buffaloes and such surgical operation “RUMEN FISTULATION” is a cruel act under the Prevention of Cruelty to Animal Act, 1960. An alternative method of rumen liquor based biodegradation is in situ method, where Agro Industrial byproducts is placed in the Nylon bag and inserted through the rumen fistula in the rumen fermentation vat (Orskov and MacDonald, 1979). There is a wide variation of end biodegradation results (in terms of In vitro dry matter and crude protein disappearance and degradability) from one series to another or from one laboratory to the other, even after vigorously standardizing the diet of the animals, the sampling conditions and the inoculum preparation (Barnes, 1967, Omed et al. 1989).

Biodegradation of Agro Industrial byproducts, municipal wastes and hospital wastes (organic materials) depends on the reproducibility of end results and such expectation is possible in only enzyme based biodegradation vessel (Jones and Hayward, 1975, MacQueen and VanSoest, 1975, and De Boever et al 1986).

It is a well-established fact that only enzyme based biodegradation vessel could yield reproducible and fermentation results. Throughout world at present crude fungal cellulase preparation (Trichoderma viride) manufactured by Sigma Aldrich family, located at USA/European subcontinent, is used as this enzyme has inherent nature of cellulolytic and proteolytic activity but India could not afford such high cost foreign exchange involved commodity for use in biodegradation fermentation vessel.

### THE OBJECT AND SUMMARY OF THE INVENTION

The object of this invention is to explore the possibility of using natural source cud from cattle and buffaloes as source of microbial enzyme for use in biodegradation fermentation vessel and thereby economize the cost of biodegradation of organic materials.

To achieve the said objective this invention provides a process of preparing cellulase and protease enzyme complex cud liquor from cattle and buffaloes cud for biodegradation of organic materials comprising:

Step 1— feeding cattle/buffaloes green leguminous/non-leguminous fodder ad libitum,

Step 2— withdrawing the feed from said cattle/buffaloes and wait till said cattle/buffalo is ruminating,

Step 3— inserting loop in the mouth of said cattle/buffaloes,

Step 4— allowing said loop till the cud is embedded on said loop,

Step 5— repeating steps 3 & 4 with different loops and different cattle/buffaloes,

Step 6— collecting said loops from the mouth of said cattle/buffaloes, and

Step 7— dipping said loops embedded with cud in at least one litre buffer nutrient solution to obtain cellulase and protease concentrate cud liquor.

The feeding of fodder is carried out for 2-3 hours.

The said loop is a fodder loop (having stem and leaves), iron loop or cotton thread loop.

The loop remains in the mouth for at lest 15-20 minutes for embedding the cud.

The buffer nutrient solution comprises mixing solution no. 1 and solution no. 2 in the ratio of 100:1 wherein:

solution no. 1 is:

a) Disodium Hydrogen phosphate—3.7 g

b) Sodium Bicarbonate 9.80 g

Mix both chemicals ‘a’ and ‘b’ and make volume—one litre with ammonia free distilled water,

and

solution no. 2 is

a) Sodium Chloride 4.7 g

b) Potassium Chloride 5.7 g

c) Calcium Chloride 0.40 g

d) Magnesium Chloride 0.60 g

Make volume 100 ml with ammonia free distilled water.

At least 4 different loops are inserted in four cattle/buffaloes to get cellulase and protease complex concentrate cud liquor.

The quantity of buffer nutrient solution depends upon the number of cattle/buffaloes used to get cellulase and protease complex concentrate cud liquor.

### THE OBJECT AND SUMMARY OF THE INVENTION

The object of this invention is to explore the possibility of using natural source cud from cattle and buffaloes as source of microbial enzyme for use in biodegradation fermentation vessel and thereby economize the cost of testing biodegradation of organic materials.

To achieve the said objective this invention provides a process of preparing cellulase and protease enzyme complex cud liquor from cattle and buffaloes cud for biodegradation of organic materials comprising:

Step 1— feeding cattle/buffaloes green leguminous/non-leguminous fodder ad libitum,

Step 2— withdrawing the feed from said cattle/buffaloes after 2-3 hours and wait till cattle/buffalo is ruminating,

Step 3— inserting fodder loop in the mouth of said cattle/buffaloes,

Step 4— allowing said loop to stay in the mouth till the cud is embedded on said loop,

Step 5— repeating steps 3 & 4 with separate fodder loops and different cattle/buffaloes,

Step 6— collecting said loops from the mouth of said cattle/buffaloes, and

Step 7— dipping said loops embedded with cud in at least one litre buffer nutrient solution to obtain cellulase and protease concentrate.

The fodder loop comprising stem and leaves of fodder.

The fodder loop remains in the mouth for at least 15-20 minutes for embedding the cud.

The buffer nutrient solution comprising mixing solution no. 1 and solution no. 2 in the ratio of 100:1 wherein:

solution no. 1 is:

a) disodium hydrogen phosphate —3.7 g

b) sodium bicarbonate 9.80 g

mix both chemicals ‘a’ and ‘b’ and make volume—one litre with ammonia free distilled water, and

solution no. 2 is

a) sodium chloride 4.7 g

b) potassium chloride 5.7 g

c) calcium chloride 0.40 g

d) magnesium chloride 0.60 g

make volume 100 ml with ammonia free distilled water.

At least four separate fodder loops are inserted in the mouth of four cattle/buffaloes to get cellulase, protease concentrate.

The quantity of buffer nutrient solution depends upon the number of cattle/buffaloes used to get cellulase, protease concentrate cud liquor.

Cellulase and protease enzyme complex cud liquor comprising inoculum consisting of cellulase impregnated undigested residue and microbial protein such that the dilution rate of the inoculum varies between 0.716 to 0.774, as compared to rumen liquor 0.550 to 0.574.

The Instant Invention is Different from the Conventional Process in Following Ways:

(i) In the cud liquor preparation, very minutely minced animal feed particles are present which are evenly distributed while in conventional process (rumen liquor) coarse animal feed particles are present. The concentration of microbial enzymes is more per unit undigested feed particles in cud liquor than rumen liquor.

(ii) In conventional rumen liquor process there is an immense need to infuse CO₂ for 20 minutes to maintain strictly anaerobic conditions, while in the instant process CO₂ infusion is not required thereby cost of processing is curtailed leading to huge saving in testing of organic materials biodegradation.

(iii) In testing, incubated material is to be separated by using sintered glass crucibles (Gl) in conventional rumen liquor based fermentation vessel while the instant invention requires Whitman filter paper No. 54 for filtering the incubated material thereby contributing to a huge saving in testing of organic materials biodegradation.

(iv) In the instant invention there is no need of maintaining modified rumen fistulated cattle and buffaloes, which is itself a very costly affair and causes environmental pollution by emission of methane and other green house gases.

Detailed description of 51 feed samples and pure nutrients taken for testing cellulolysis and proteolysis potential of cud liquor and biodegradability is given below:

Batch I: (India)

Groundnut cake, Mustard cake, Soybean meal, Fish meal, Cotton seed, Cotton seed cake, Maize, Barley, Rice polish and Gram churi

Batch II: (India)

Mustard Cake —10 different locations samples, Cotton Seed cake —10 different locations samples

Batch III: (Germany)

Mustard cake—10 different locations samples, Soybean Meal—8 different locations samples, Maize Gluten Feed—3 different locations samples.

Batch IV:

Sigma Cellulose type 101, (catalogue no. S-6790)

Batch V: Raw Casein, Protected Casein

Steps for Testing Animal Feed Samples Given in Batch I, II & III, Cellulose in Batch IV and Raw Casein, Protected Casein in Batch V

The following steps are applicable for each batch.

Testing for Batch I:

Step 1: 250 mg of feed material is taken in a test tube and 25 ml cud liquor is added to said feed material. The said tube is then kept in oven for 48 hours.

Step 2: three blank tubes with 25 ml cud liquor in each tube is taken and kept in the oven for 48 hours,

Step 3: 25 ml of Std. Sigma cellulase enzyme is taken in each three different tubes and is kept in the oven for 48 hours,

Step 4: after 48 hours of incubation 0.2 g of pepsin and 2 ml of 6N HCl is added to each tube and is incubated further for 48 hours,

Step 5: centriftiging the incubated material in the tube for 15 minutes at 2000 gm speed,

Step 6: allowing the test tube to stand for a while and filter with Whatman Filter paper no. 54.

Step 7: washing the residue 6 times with warm water

Step 8: removing the filter paper with residue and place in the oven for 12 hours at 85° C.

Step 9: thereafter, keeping the filter paper with residue in desiccator

Step 10: weighing the filter paper and residue three times at 2 hours interval.

Step 11: taking the average by eliminating the weight of empty filter paper to get the percentage of biodegradation.

Step 12: at the time of weighing of sample, 2 gm of substrate is taken separately in moisture cup and kept in the oven for 24 hours at 80° C. for estimating dry matter in the incubated samples, in order to calculate the weight of incubated material on dry matter basis,

Step 13: weigh 1 gm of raw material for nitrogen estimation and preserve undigested residue with filter paper for nitrogen estimation in order to calculate the biodegradation of protein in the testing material by Kjeldahl method.

The above testing procedure is followed for batch II, III, IV & V also. The amount of sample taken in Batch II & III is 250 mg and 60 mg of Sigma Cellulose type 101 is taken in Batch IV. In Batch V, 150 mg of raw and protected casein is taken for testing. The incubation period for testing Sigma Cellulose type 101 in Batch IV is 240 hours instead of 96 hours.

Calculations

$$\frac{\begin{matrix}\begin{matrix}In vitro dry matter disappearance per cent ( IVDMD \% ) \\ Samples dry matter weight - residual dry matter weight -\end{matrix} \\ residual dry matter weight of blank\end{matrix}}{Samples dry matter weight} \times 100 \qquad ( a )$$

(b) Effective Ruminal dry matter/protein degradability percent (ED %)

(Orskov and MacDonald, 1979)

$$P = ( a + \frac{( b x c )}{( c + k )} ) \times 100$$

P=Proportion of dry matter/protein degraded

a=Cold water extractable N/DM as decimal of total N/DM

b=Slowly degradable N/DM as decimal of total N/DM

c=Rate of change constant for “b” fraction or degradation rate

k=Ruminal passage rate or ruminal out flow rate

Results

The results related to the biodegradability of dry matter/protein with respect to Mustard cake (India & Germany), Soyabean meal (India and Germany) and Cotton seed (India) as well as other agro-industrial byproducts are calculated and are given in Tables 1 to 4 for all the 51 samples of 11 different agro-industrial byproducts.

The efficacy of biodegradation fermentation vessel is measured in terms of In vitro dry matter/protein disappearance and degradability and data are presented in Tables 1 to 4. It is quite clear from the perusal of data that cud liquor based fermentation vessel yielded results of In vitro dry matter/protein disappearance and degradability at par with the conventional rumen liquor based conventional situ method, as published in an internationally recognised book (AFRC, 1993). In the case of Mustard Cake, effective degradability percent of crude protein (ED %) assessed by In situ method (Sampath, 1990) was 69, while the instant invention based fermentation vessel yielded 74 and 72 ED % using cattle/buffaloes cud liquor respectively. Another testing material Cottonseed Cake also yielded results of ED % on same lines confirming required activity of cellulase and protease in cud liquor of instant invention.

The data related to In vivo nitrogen degradability percent estimates for Soyabean meal, Groundnut cake and Fish meal based on In vivo study are compared with the figures obtained by cud liquor (cattle/buffalo) based fermentation vessel of instant invention. It is worth noting that cellulase and protease present in cud liquor is equally effective in biodegrading Agro Industrial by products as it has been reported on the basis of In vivo study presented in Table 5.

Table 6 shows the proteolysis potential of Cud Liquor in terms of casein crude protein disappearance percent was significantly (P<0.05) higher as compared to rumen liquor (Score card value 153 vis-a-vis 133 in cattle) and (Score card value 172 vis-a-vis 153 in Buffalo), overall proteolysis score card values are significantly (P<0.05) higher in Buffalo as compared to cattle when protected casein was tested (172 vis-a-vis 153 with cud liquor) and (153 vis-a-vis 133) with rumen liquor. Higher score card values with cud liquor and rumen liquor as compared to Fungal cellulase is a documentary evidence of presence of higher concentration of proteases complex with earlier incubation media

It may be concluded from the present study that cud liquor is a very rich source of proteases complex having excellent proteolysis potential and its commercial exploitation is feasible being cheaper natural source that too toxin free. Ruminant (Cow and Buffalo) could be prevented from Cruel Act of Rumen fistulation by using the newly innovated process of preparing CUD

Liquor

In table 7, against the standard known value of 93.6 units of cellulase present in 15 ml of incubation media finally prepared fungal cellulase (Sigma Cat. No. C-9422)—T₅, concentration of cellulase was 69.233, 79.449, 75.970 and 76.582 units, respectively in T, (cud liquor—cattle), T₂ (cud liquor —Buffalo), T₃ (Rumen liquor—cattle) and T₄ (Rumen liquor—Buffalo), respectively. The cost of cellulase present in 15 ml media as per Sigma catalogue is 0.55, 0.63, 0.61, 0.61 and 0.75 US Dollar in treatments T₁, T₂, T₃, T₄ and T₅, respectively. Indian currency equivalent comes to Rs. 27.5, 31.5, 30.5, 30.5 and 37.5, respectively under treatments, T₁, T₂, T₃, T₄ and T₅ respectively. It is clear from these calculation that there is a net saving of US Dollar 0.59 (India Rs. 29.5), respectively through a cud liquor based cellulase plus protease complex process proposed for patenting. The additional advantage of this process is due to the presence of protease complex, while this enzyme complex is missing from proprietary preparation Sigma cat. No. C-9422 based on Fungal cellulase. As cud from cow and buffalo is toxin free therefore, good results of biodegradation of organic materials are expected from this new process.

Table 8 shows cellulolytic potential of Cud Liquor (Buffalo)—T₂ is at par with Rumen Liquor (Buffalo)—T₄, however Cud Liquor (Cattle)—T, showed significantly (P<0.05) less cellulolytic potential as compared to Cud Liquor (Buffalo), but the values are within a reasonable limit of cellulose disappearance percent.

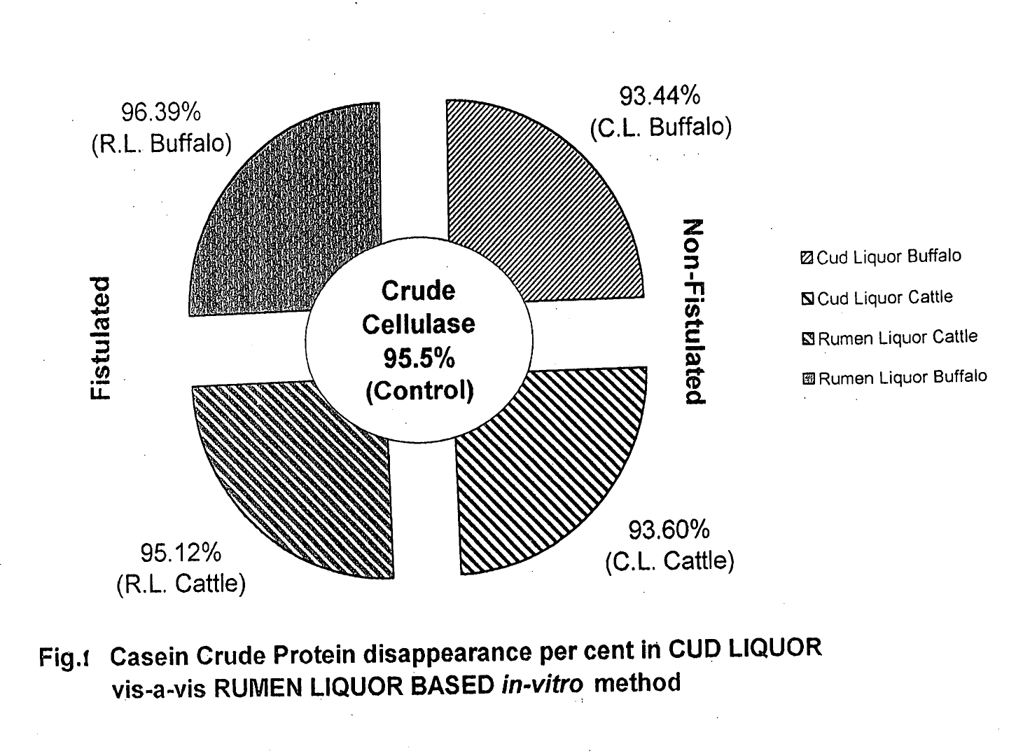

The proteolytic activity of gram negative bacilli enriched cud liquor (cattle/buffalo) vis-a-vis rumen liquor (cattle/buffalo) was tested using raw casein protein (98.5% CP) and results of crude protein disappearance are presented through pie diagram, as shown in FIG. 1 of the accompanying drawings. The overall value of casein crude protein disappearance percent under In vitro fermentation vessel based on cellulase (Trichoderma Viride), cud liquor (cattle/buffalo), rumen liquor (cattle/buffalo) was 95.51±0.597, 93.60±0.344, 93.44±0.639, 95.1±0.748, 95.39±0.518, respectively. In general, casein protein disappearance was significant (P<0.05) higher with rumen liquor as compared to cud liquor, but the values in both the cases are within the range of published figures (Sampath, 1990).

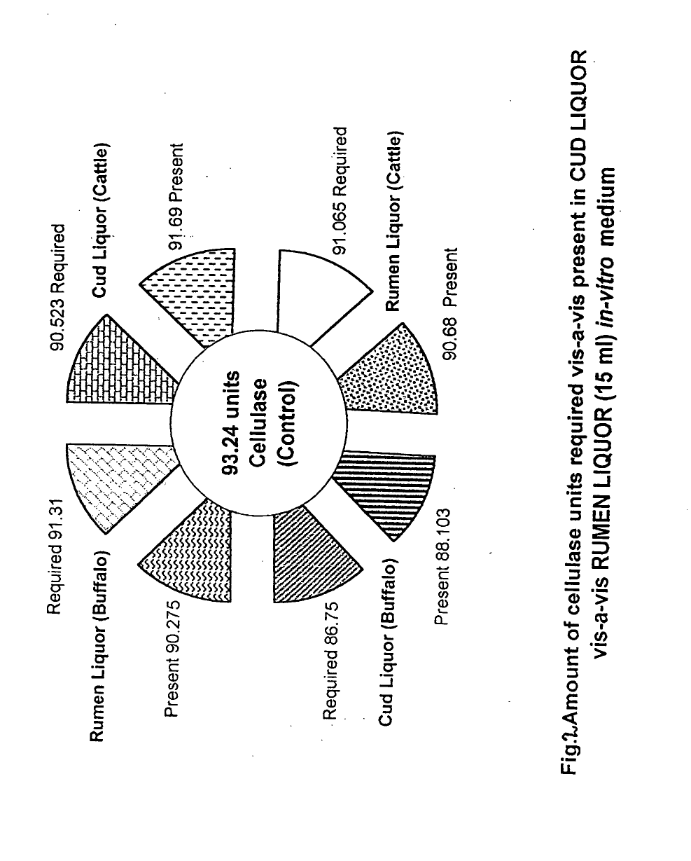

The concentration of cellulase unit (one unit will liberate 1.0 micro mole of glucose from cellulose in one hour at pH 5.0 at 37° C., 02 hrs incubation time) in cud liquor vis-a-vis rumen liquor are presented in pie diagram, as shown in FIG. 2. It was observed that concentration of cellulase in cud liquor and rumen liquor was within the requirement limit.

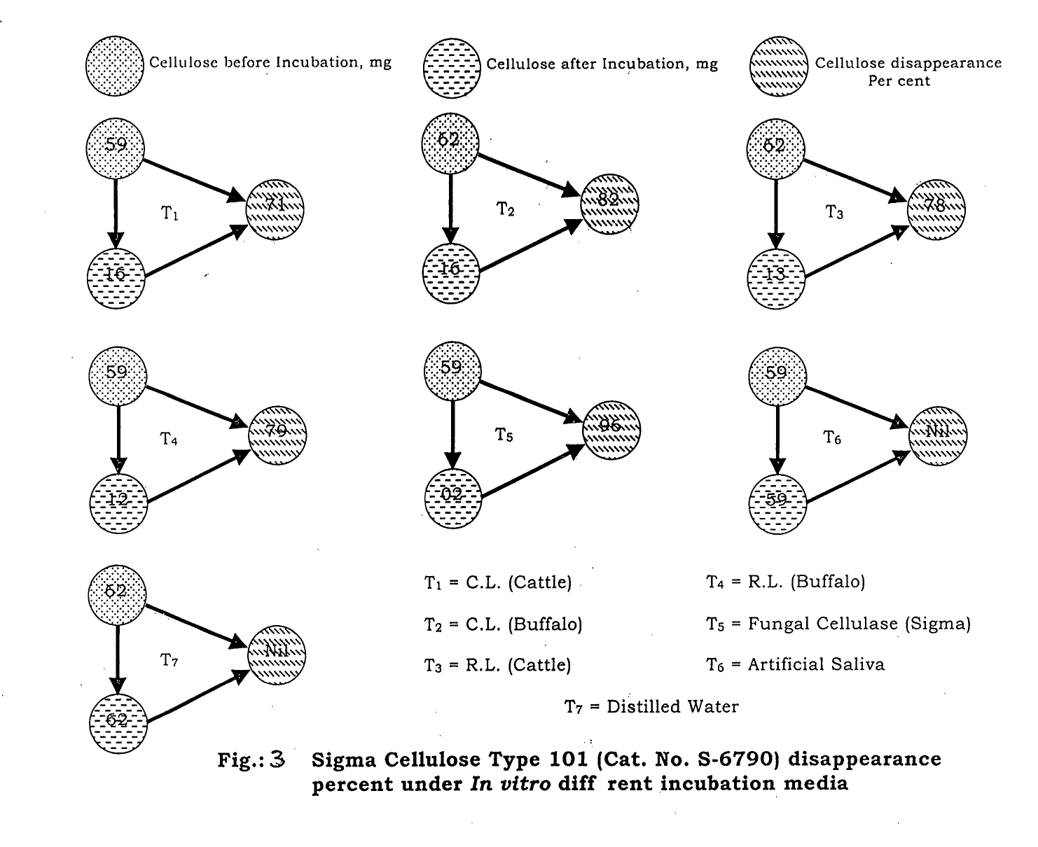

In general, in FIG. 3, Sigma cellulose, Type 101 (Cat. No. S-6790) disappearance percent under in vitro different incubation media was 71, 82, 78, 79, 96, Nil, and Nil in treatments T₁, T₂, T₃, T₄, T₅, T₆ and T₇ respectively. By taking consideration of actual value of T₅ (96.51) as 100, then Score card values of cud liquor (cattle)—T₁, Cud Liquor (Buffalo)—T₂, Rumen Liquor (Cattle)—T₃ and Rumen Liquor (Buffalo)—T₄ were 74, 85, 81 and 81, respectively.

It may be concluded from the present study that cud liquor with inoculum dilution rate (0.774 and 0.716) could provide enough concentration of cellulase required for cellulolysis (156 units/25 ml buffer) of organic materials with expected efficiency of conventional rumen liquor. Its commercial exploitation is possible being cheaper natural source that too toxin free. Ruminant (Cow and Buffalo) could be prevented from Cruel Azt of Rumen Fistulation by using the newly innovated process of preparing CUD

Liquor.

A prediction equation was fitted between dry matter (x) and crude protein (y) using data obtained by cud liquor (cattle/buffalo) based fermentation vessel, overall turnover (b) was 0.395 (cattle) and 0.437 (buffalo), yielding prediction percent (y) value 101 (cattle) and 99.79 (buffalo).

Cattle, Y=51.434+0.395X,r²=0.608,Sxy%=2.970

Buffaloes, Y=50.350+0.437X,r²=0.643,Sxy%=3.323

The value of prediction percent indicate the fitness of cud liquor based fermentation vessel for testing biodegradation of organic materials.

Advantages of the Instant Invention

1. Cud from cattle and buffaloes is an inexpensive natural source of crude cellulase and protease complex and could replace imported fungal cellulase (Trichoderma Viride) preparation from Sigma, Sweden.

2. The cud cellulase and protease complex could be used in bioremediation process.

3. Cud liquor may be used as rumen modifiers—optimize rumen function specially under acidosis and anorexia conditions and pharmaceutical industry could make use of this invention and capsule after lypholisation and ultrafiltration could be prepared for treating Anorexia suffering cattle/buffaloes.

4. This invention is an alternative of rumen fistulation which is banned by Ministry of Social Justice and Empowerment, Government of India, vide notification dated Dec. 15, 1998 under the prevention of cruelty to Animal Act, 1960 (59 of 1960) and provide valuable solution to protect the cattle and buffaloes from the cruel act of rumen fistulation.

5. Cud liquor (cattle/buffalo) having cellulase+protease complex could be used as growth promoter in the Swine ration and present invention provides substitute of imported growth promoter.

6. Cud liquor (cattle/buffalo) could work as a flavouring media for enhancing feed intake of Animal Feed in mash form, pellet form or compact feed block. This is a good substitute of imported costly flavouring agent.

TABLE 1
Ruminal Protein degradability, Crude Protein (CP) Rumen degradable protein (RDP) and
undegradable protein (UDP) Content of Indian & Germany Animal feeds

| | | Crude | | | | | | | | |
|---|---|---|---|---|---|---|---|---|---|---|
| | | Protin | | | | | | | | |
| S. | | g/kg/ | | | RDP g/Kg DM | | UDP g/kg/DM | | AFRC | Sampath |
| No. | Feedstuffs | DM | Cattle | Buffalo | Cattle | Buffalo | Cattle | Buffalo | (1993) | (1990) |
| 1. | Gram Churi | 238.4 | dg8(0.52) | dg8(0.55) | 123.97 | 131.12 | 114.43 | 107.28 | — | — |
| 2. | Soybean meal | 416.2 | dg5(0.66) | dg5(0.66) | 274.69 | 274.69 | 141.51 | 141.51 | dg8³(0.60) | 0.66 |
| | (India) | | | | | | | | | |
| | (Germany) | 568.8 | ″ | ″ | 375.41 | 375.41 | 193.39 | 193.39 | ″ | ″ |
| 3. | Cottonseed | 268.7 | dg8(0.57) | dg8(0.56) | 153.16 | 150.47 | 115.54 | 118.23 | dg8(0.59) | 0.51 |
| | Cake | | | | | | | | | |
| 4. | Rice Polish | 134.1 | dg8(0.65) | dg8(0.65) | 87.16 | 87.16 | 46.94 | 46.94 | dg6(0.66) | 0.45 |
| 5. | Mustard Cake | 362.72 ± | dg8(0.74) | dg8(0.72) | 268.41 | 261.16 | 94.31 | 101.56 | dg8(0.69) | 0.75 |
| | (India) | 4.336 | | | | | | | | |
| | (Germany) | 438.95 ± | dg8(0.72) | dg8(0.72) | 316.04 | 316.04 | 122.91 | 122.91 | dg8³(0.73) | 0.69 |
| | | 8.588 | | | | | | | | |
| 6. | Maize | 115.1 | dg8(0.25) | dg8(0.32) | 28.77 | 36.83 | 86.33 | 78.27 | dg8(0.31) | — |
| 7. | Barley | 102.3 | dg8(0.86) | dg8(0.86) | 87.98 | 87.98 | 14.32 | 14.32 | dg8(0.82) | 0.82 |
| 8. | Groundnut | 423.4 | dg5(0.69) | dg5(0.69) | 292.14 | 292.14 | 131.26 | 131.26 | dg6(0.69) | 0.68 |
| | Cake | | | | | | | | | |
| 9. | Cottonseed | 207.0 | dg8(0.54) | dg8(0.61) | 111.78 | 126.27 | 95.22 | 80.73 | — | — |
| 10. | Fish meal | 428.4 | dg8(0.31) | dg8(0.32) | 132.80 | 137.09 | 295.60 | 291.31 | dg8(0.42) | 0.41 |
| 11. | Maize gluten | 245.833 | dg(0.70) | dg5(0.69) | 172.08 | 169.62 | 73.75 | 76.21 | dg6(0.69) | 0.75 |
| | Feed | | | | | | | | | |

TABLE 2
Ruminal Dry matt r and protein degradation of some feed ingradients
(FIGS. obtained by C.L. based in vitro method vis-à-vis values of other m thods)

| | | | | | | | Value (Dm-“b”, |
|---|---|---|---|---|---|---|---|
| Sr. | | | | | Incubation | | CP-“b” DM-ED |
| N . | Author | Year | Journal | Method | (hrs.) | Observation | % and CP-ED%) |

MUSTARD CAKE

| 1. | Negi et al. | 1989 | Indian J. Animal | Nylon Bag | 48 | N-ED% | 84.1 | |
|---|---|---|---|---|---|---|---|---|
| | | | Nutrition 6: 1-12 | | | | | |
| 2. | Sharma and Singh | 1997 | Indian J. Animal | ″ | 48 | DM-“b” | 72.86 | |
| | | | Nutrition 14: | | | N-“b” | 87.59 | |
| | | | 213-222 | | | DM-ED% | 64.54 | |
| | | | | | | N-ED% | 69.81 | |
| 3. | Sinha and Singhal | 1997 | Indian J. Animal | ″ | 36 | Oil Cake | | |
| | | | Nutrition 14: | | | DM-ED% | 53.2 | |
| | | | 223-228 | | | CP-ED% | 61.8 | |
| | | | | | | Solvent | | |
| | | | | | | Extacted | | |
| | | | | | | DM-ED% | 58.9 | |
| | | | | | | CP-ED% | 69.8 | |
| 4. | Sampath, K. T. | 1990 | Indian J. Dairy | Nylon | — | CP-ED% | 69.0 | |
| | | | Sci. 43: 1-9 | bag, | | | | |
| | | | | in vivo | | | | |
| 5. | Agricultural Food | 1993 | Energy and | Nylon bag | — | CP-ED% | 73.0 | |
| | Research Council, | | Protein | | | CP-“b” | 75.0 | |
| | U.K. | | requirements of | | | | | |
| | | | Ruminants, CAB | | | | | |
| | | | International | | | | | |
| | | | (UK) | | | | | |
| 6. | Van Straalen & | 1990 | Feed stuffs | Nylon bag | 48 | CP-ED% | 66.0 | |
| | Tamminga | | Evaluation Publ. | | | | | |
| | | | Butterworths (UK) | | | | | |
| 7. | Present Study | | In vitro | Cattle | 48:48 | | C | B |
| | | | (Cud Liquor) | Buffalo | | India | | |
| | | | | | | DM-“b” | 67.0 | 66.0 |
| | | | | | | CP-“b” | 77.0 | 83.0 |
| | | | | | | DM-ED% | 71.36 | 71.65 |
| | | | | | | CP-ED% | 74.00 | 72.00 |
| | | | | | | Germany | | |
| | | | | | | DM-“b” | 72.78 | 67.20 |
| | | | | | | CP-“b” | 82.00 | 82.00 |
| | | | | | | DM-ED% | 69.03 | 71.01 |
| | | | | | | CP-ED% | 72.00 | 72.00 |

1. D.M.-“b”, per cent dry matter disappeared
2. C.P.-“b”, per cent crude protein disappeared
3. D.M.-ED%, Dry matter effective degradability per cent
4. C.P.-ED%, Crude protein effective degradability per cent

TABLE 3
Ruminal Dry matter and protein degradation of some feed ingradients
(FIGS. obtained by C.L. based in vitro method vis-à-vis values of other methods.

| Sr. | | | | | Incubation | | CP-“b” DM-ED |
|---|---|---|---|---|---|---|---|
| No. | Author | Year | Journal | Method | (hrs.) | Observation | % and CP-ED%) |
| 1. | Sharma & | 1997 | Indian J. Animal | Nylon Bag | 48 | DM-“b” | 41.22 |
| | Singh | | Nutrition 14: | | | N-“b” | 57.62 |
| | | | 213-222 | | | DM-ED% | 30.44 |
| | | | | | | N-ED% | 38.43 |
| 2. | Garg, M. R. & | 1994 | Indian J. Dairy | ″ | 24 | DM-“b” | 54.03 |
| | Gupta, B. N. | | Science 47: | | | CP-“b” | 60.13 |
| | | | 162-165 | | | CP-ED% | 55.51 |
| 3. | Sampath, K. T. | 1990 | Indian J. Dairy | Nylon Bag | — | CP-ED% | 51.00 |
| | | | Science 43: 1-9 | In vivo | | | |
| 4. | Chaturvedi and | 1995 | Indian J. Anim. | Nylon Bag | 40 | DM-“b” | 63.68 |
| | Wali | | Nutrition 12: | | | CP-“b” | 64.94 |
| | | | 133-139. | | | DM-ED% | 41.60 |
| | | | | | | CP-ED% | 49.20 |
| 5. | AFRC | 1993 | Energy and | Nylon bag | — | CP-ED% | 59.00 |
| | | | Protein | | | CP-“b” | 76.00 |
| | | | requirements of | | | | |
| | | | Ruminants, CAB | | | | |
| | | | International | | | | |
| | | | (UK) | | | | |
| 6. | Van Straalen & | 1990 | Feed stuffs | Nylon bag | 48 | CP-ED% | 57.0 |
| | Tamminga | | Evaluation Publ. | | | | |
| | | | Butterworths (UK) | | | | |

| | | | | | | | |
|---|---|---|---|---|---|---|---|
| 7. | Present Study | In vitro | Cattle | 48:48 | | C | B |
| | | (Cud Liquor) | Buffalo | | | | |
| | | | | | DM-“b” | 43.80 | 40.78 |
| | | | | | CP-“b” | 75.00 | 76.00 |
| | | | | | DM-ED% | 81.35 | 82.64 |
| | | | | | CP-ED% | 58.00 | 57.00 |

TABLE 4
Ruminal Dry matter and pr tein degradation of some feed ingradients
(FIGS. obtained by C.L. based in vitro method vis-à-vis values of other methods)

| | | | | | | | Value (Dm-“b”, |
|---|---|---|---|---|---|---|---|
| Sr. | | | | | Incubation | | CP-“b” DM-ED |
| No. | Author | Year | Journal | Method | (hrs.) | Observation | % and CP-ED%) |

SOYBEAN MEAL

| 1. | Negi et al. | 1989 | Indian J. Animal | Nylon Bag | 48 | N-ED% | 38.4 | |
|---|---|---|---|---|---|---|---|---|
| | | | Nutrition 6: 1-12 | | | | | |
| 2. | Sampath, K. T. | 1990 | Indian J. Dairy | Nylon Bag | — | CP-ED% | 66.0 | |
| | | | Science 43: 1-9 | In vivo | | | | |
| | | | | In vivo | | | | |
| 3. | AFRC | 1993 | Energy and | Nylon bag | — | CP-ED% | 61.0 | |
| | | | Protein | | | CP-“b” | 92.0 | |
| | | | requirements of | | | | | |
| | | | Ruminants, CAB | | | | | |
| | | | International | | | | | |
| | | | (UK) | | | | | |
| 4. | Van Straalen & | 1990 | Feed stuffs | Nylon bag | 48 | CP-ED% | 61.0 | |
| | Tamminga | | Evaluation Publ. | | | | | |
| | | | Butterworths (UK) | | | | | |
| 5. | Present Study | | In vitro | Cattle | 48:48 | India | C | B |
| | | | (Cud Liquor) | Buffalo | | DM-“b” | 87.83 | 88.58 |
| | | | | | | CP-“b” | 88.00 | 88.00 |
| | | | | | | DM-ED% | 62.71 | 61.98 |
| | | | | | | CP-ED% | 66.00 | 66.00 |
| | | | | | | Germany | | |
| | | | | | | DM-“b” | 94.23 | 85.37 |
| | | | | | | CP-“b” | 91.00 | 94.00 |
| | | | | | | DM-ED% | 59.89 | 63.49 |
| | | | | | | CP-ED% | 65.00 | 63.00 |

TABLE 5
Nitrogen d gradability (%) stimates for soyb an m al, groundnut cake and fish m al
(Comparison of In-vivo* vis-à-vis In-vitro C.L. based method)

| | | | | | In-vitro cud liquor | | In-vivo value |
|---|---|---|---|---|---|---|---|
| | | In-vivo Method | | | (48 h:48 h) | | based on steer |
| Sr. | | Duodenal | Ammonia | Polyester | | | and lamb growth |
| No. | Name | flow* | kinetics* | bag* | Cattle | Buffalo | trials** |
| 1. | Soybean meal | | | | | | |
| | (U.K.) | 88 | 84 | 82 | — | — | — |
| | (India) | — | — | — | 82 (dg2) | 82 (dg2) | — |
| | (Germany) | — | — | — | 81 (dg2) | 81 (dg2) | — |
| | (USA) | — | — | — | — | — | 70 |
| 2. | Groundnut cake | | | | | | |
| | (U.K.) | 76 | 54 | 67 | — | — | — |
| | (India) | — | — | — | 69 (dg5) | 69 (dg5) | — |
| 3. | Fish meal (U.K.) | 57 | 45 | 60 | — | — | — |
| | (India) | — | — | — | 56 (dg2) | 57 (dg2) | — |

*SIDDONS, R. C., PARADINE, J., GALE, D. L. and EVANS, R. T. (1985) Estimation of the degradability of dietary protein in the sheep rumen by in vivo and in vitro procedures. British Journal of Nutrition 54: 545-561.
**MARY, P. F., KLOPFENSTEIN, T. and BRITTON, R. A. (1985) Evaluation of laboratory Techniques for predicting Ruminal Protein degradation. J. Dairy Sci., 68: 829-39.

TABLE 6
Casein crude protein disappearance percent under different in vitro media
(raw casein vis-à-vis protected casein)

| Sr. | | | Raw Casein* | | Protected Casein** | |
|---|---|---|---|---|---|---|
| No. | In vitro media | Replicate | Cattle | Buffalo | Cattle | Buffalo |
| 1 | Cud Liquor | 03 | 93.606^{a} ± 0.314 | 93.44^{a} ± 0.583 | 74.616^{b} ± 1.256 | 84.209^{c} ± 2.011 |
| | | | (98.003) | (97.829) | (152.748) | (172.386) |
| 2 | Rumen Liquor | 03 | 95.12^{a} ± 0.683 | 96.39^{a} 0.473 | 64.822^{b} ± 2.864 | 74.537^{c} ± 0.715 |
| | | | (99.588) | (100.918) | (132.698) | (152.586) |

| | | | | |
|---|---|---|---|---|
| 3 | Fungal Cellulase Sigma | 03 | 95.513^{a} ± 0.545 | 48.849^{b} 1.364 |
| | (Cat No. E-9422) | | (100) | (100) |
| 4 | Distilled water | 03 | 98.256^{a} ± 0.433 | 11.206^{b} ± 1.397 |

** Figures in parenthesis indicate score card value
^{a,b,c}means in the same row followed by different letters are different (P < 0.05)

TABLE 7
Cost of cellulase present in 15 ml incubation media based on Fungal cellulase (Sigma Cat. No.
C-9422) vis-à-vis Rumen liquor and Cud liquor

| | | Cellulase units | Sigma cellulose | Cost in US Dollar | Cost in Indian Rs. |
|---|---|---|---|---|---|
| | | present in 15 ml | Type 101 (Cat. | of cellulase | of Cellulas |
| | Incubation media | incubation | No. S-6790) | present in 15 ml | present in 15 ml |
| | (15 ml for single | media for single | disappearance | media for single | media for single |
| Tr atments | sample) | sample | per cent | sample | sample |
| T₅ | Fungal cellulase (Sigma | 93.6 | 96.51 | 0.75 | 37.5 |
| | cat. No. E-9422) | | | | |
| T₄ | Rumen liquor (Buffalo) | 76.582 | 78.963 | 0.61 | 30.5 |
| T₃ | Rumen liquor (Cattle) | 75.970 | 78.333 | 0.61 | 30.5 |
| T₂ | Cud liquor (Buffalo) | 79.449 | 81.920 | 0.63 | 31.5 |
| T₁ | Cud liquor (Cattle) | 69.233 | 71.386 | 0.55 | 27.5 |

N.B. 70-80 per cent disappearance of Sigma cellulose Type 101 (Cat. No. S-6790) is normal value as per published. Literature during 10 days incubation.

TABLE 8
Sigma Cellulose, Type 101 (Cat. No. S-6790) disappearance per cent under
In vitro different incubation media

| | Treatments | | | | | | |
|---|---|---|---|---|---|---|---|
| | Cud | Cud | Rumen | Rumen | Fungal | | |
| | Liquor | Liquor | Liquor | Liquor | Cellulase | Artificial | Distilled |
| Sr. | (Cattle) | (Buffalo) | (Cattle) | (Buffalo) | (Sigma) | Saliva | water |
| No. | T₁ | T₂ | T₃ | T₄ | T₅ | T₆ | T₇ |
| I | 71.42 | 75.0 | 78.46 | 78.57 | 98.46 | 1.78 | Nil |
| II | 64.28 | 84.61 | 81.54 | 81.54 | 92.86 | Nil | Nil |
| III | 78.46 | 86.15 | 75.00 | 76.78 | 98.21 | Nil | Nil |
| Mean ± | 71.386^{b, c} ± | 81.92^{b, d} ± | 78.333^{b, e} ± | 78.963^{b, e} ± | 96.51^{a} ± | Nil | Nil |
| SEm | 4.093 | 3.488 | 1.889 | 1.388 | 1.826 | | |

^{a, b}means in the same row followed by different letters are different (P < 0.05)

We claim:

1. A process of preparing cellulase and protease enzyme complex cud liquor from cattle and buffaloes cud for biodegradation of organic materials comprising:
Step 1— feeding cattle/buffaloes green leguminous/non-leguminous fodder ad libitum,
Step 2— withdrawing the feed from said cattle/buffaloes after 2-3 hours and wait till cattle/buffalo is ruminating,
Step 3— inserting fodder loop in the mouth of said cattle/buffaloes,
Step 4— allowing said loop to stay in the mouth till the cud is embedded on said loop,
Step 5— repeating steps 3 & 4 with separate fodder loops and different cattle/buffaloes,
Step 6— collecting said loops from the mouth of said cattle/buffaloes, and
Step 7— dipping said loops embedded with cud in at least one litre buffer nutrient solution to obtain cellulase and protease concentrate.

2. A process as claimed in claim 1 wherein the fodder loop comprising stem and leaves of fodder.

3. A process as claimed in claim 1 wherein the fodder loop remains in the mouth for at least 15-20 minutes for embedding the cud.

4. A process as claimed in claim 1 wherein the buffer nutrient solution comprising mixing solution no. 1 and solution no. 2 in the ratio of 100:1 wherein:
solution no. 1 is:
c) disodium hydrogen phosphate —3.7 g
d) sodium bicarbonate 9.80 g
mix both chemicals ‘a’ and ‘b’ and make volume—one litre with ammonia free distilled water,
and
solution no. 2 is
e) sodium chloride 4.7 g
f) potassium chloride 5.7 g
g) calcium chloride 0.40 g
h) magnesium chloride 0.60 g
make volume 100 ml with ammonia free distilled water.

5. A process as claimed in claim 1 wherein at least four separate fodder loops are inserted in the mouth of four cattle/buffaloes to get cellulase, protease concentrate.

6. A process as claimed in claim 1 wherein the quantity of buffer nutrient solution depends upon the number of cattle/buffaloes used to get cellulase, protease concentrate cud liquor.

7. Cellulase and protease enzyme complex cud liquor comprising inoculum consisting of cellulase impregnated undigested residue and microbial protein such that the dilution rate of the inoculum varies between 0.716 to 0.774, as compared to rumen liquor 0.550 to 0.574.
